(19) United States
(12) Patent Application Publication
(10) Pub. No.: US 2018/0071733 A1
(43) Pub. Date: Mar. 15, 2018

(54) PRESSURIZABLE CARTRIDGE FOR POLYMERASE CHAIN REACTIONS

(71) Applicant: DxNA LLC, St. George, UT (US)

(72) Inventors: Ray Cracauer, St. George, UT (US); Clark Braten, St. George, UT (US); William D. Bickmore, St. George, UT (US); Doyle Hansen, St. George, UT (US); Ernie Sumsion, St. George, UT (US); Frank Spangler, St. George, UT (US); Brent L. Weight, St. George, UT (US); Darryl K. Zitting, St. George, UT (US)

(21) Appl. No.: 15/812,104

(22) Filed: Nov. 14, 2017

Related U.S. Application Data
(63) Continuation-in-part of application No. 13/813,882, filed on Jun. 10, 2013, now Pat. No. 9,816,131.
Application No. 13/813,882 is a continuation-in-part of application No. 14/455,542, filed on Aug. 8, 2014, now abandoned.
Application No. 14/455,542 is a continuation-in-part of application No. 14/134,736, filed on Dec. 19, 2013, now abandoned.
(60) Provisional application No. 61/369,925, filed on Aug. 2, 2010.
(60) Provisional application No. 61/739,611, filed on Dec. 19, 2012.

(51) Int. Cl. B01L 3/00; C12Q 1/68

(52) U.S. Cl. CPC B01L 3/502715; B01L 3/502723; C12Q 1/686; B01L 3/5029; B01L 2400/0478; B01L 2300/022; B01L 2300/0681; B01L 2300/123; B01L 2200/0684

(57) ABSTRACT

A sample processing module includes a PCR assembly including at least one PCR reaction vial. A sample assembly is configured to hold a fluid sample therein, said sample assembly being attachable to said PCR assembly so as to form a closed system. A delivery channel is formed in said PCR assembly, said delivery channel being in fluidic communication with said PCR reaction vial and with said sample assembly. A pressurization device, said pressurization device being configured to increase a pressure of said fluid sample in said sample assembly and direct at least a portion said fluid sample through said delivery channel to said PCR reaction vial. A chemically inert magnetically responsive bead is disposed within the PCR reaction vial. A system is provided for oscillating for altering a position of the magnetically responsive bead within the PCR reaction vial.

### PRIORITY CLAIM

This application is a continuation-in-part of U.S. patent application Ser. No. 13/813,882, filed Jun. 10, 2013, which claims the benefit of U.S. Provisional Application No. 61/369,925, filed Aug. 2, 2010; and is a continuation-in-part of U.S. patent application Ser. No. 14/455,542, filed Aug. 8, 2014, which is a continuation-in-part of U.S. patent application Ser. No. 14/134,736, filed Dec. 19, 2013, which claims priority to U.S. Provisional Patent Application Ser. No. 61/739,611, filed Dec. 19, 2012; all of which are hereby incorporated herein by reference in their entirety.

### BACKGROUND OF THE INVENTION

The present invention is directed to the field of thermocyclers used in the practice of the polymerase chain reaction (PCR).

### BRIEF DESCRIPTION OF THE DRAWINGS

Additional features and advantages of the invention will be apparent from the detailed description which follows, taken in conjunction with the accompanying drawings, which together illustrate, by way of example, features of the invention:

FIG. 1 is a perspective view of an embodiment of a sample processing module;

FIG. 2 is a side view of a sample assembly;

FIG. 3 is a cross-sectional side view taken along section line 3-3 of FIG. 2;

FIG. 4 is a side view of the sample assembly of FIG. 2 and a swab;

FIG. 5 is another side view of the sample assembly of FIG. 2 and a swab;

FIG. 6 is a perspective view of a PCR assembly;

FIG. 7 is a top view of the PCR assembly of FIG. 6;

FIG. 8 is a cross sectional side view of the PCR assembly of FIG. 6;

FIG. 9 is a bottom view of the PCR assembly of FIG. 6;

FIG. 10 is a side view of the sample processing module of FIG. 1 and optional associated structures;

FIG. 11 is a partial cross-sectional view of an embodiment of the PCR assembly of FIG. 6;

FIG. 12 is a partial top view of the lenses and surrounding components of the PCR assembly of FIG. 6;

FIG. 13 is a bottom view corresponding to FIG. 12;

FIG. 14 is a cross-sectional view of another embodiment of a PCR assembly;

FIG. 15 is a cross-sectional side view of another embodiment of a sample assembly taken along section line 3-3 of FIG. 2;

FIG. 16 is a cross-sectional view of a first embodiment of a magnetically responsive mixing bead capable of use within a mixing apparatus in accordance with an embodiment of the present invention;

FIG. 17 is a cross-sectional view of a second embodiment of a magnetically responsive mixing bead capable of use within a mixing apparatus in accordance with an embodiment of the present invention;

FIGS. 18A-18D are side views depicting a closed reaction well in accordance with an embodiment of the present invention containing a magnetically responsive mixing bead; various levels of solutions and reagents are shown in the various figures;

FIGS. 19A-19B are perspective, partially schematic views depicting various positions of a magnet with respect to the reaction well and how a corresponding magnetic field may affect the position of the mixing bead;

FIGS. 19C-19D are perspective, partially schematic views depicting various positions of a magnet with respect to the reaction well and how a corresponding magnetic field may affect the position of the mixing bead, with the magnet being positioned off-axis relative to an optics measurement system directed into a top of the reaction well;

FIGS. 20A-20B are perspective, partially schematic views depicting positioning of a plurality of magnets with respect to the reaction well and how this may induce movement of the mixing bead within the reaction well at increased speeds;

FIGS. 21A-21B are perspective, partially schematic views depicting an electromagnet being used to induce movement of the mixing bead within the reaction well;

FIGS. 22A-22B are perspective, partially schematic views depicting a plurality of electromagnets being positioned about the reaction well in order to induce movement of the mixing bead within the reaction well at increased speeds;

FIGS. 23A-23C are perspective, partially schematic views depicting a mechanically displaced electromagnet configured to move the bead in accordance with one aspect of the present invention which utilizes magnets and magnetomotive force to move the electromagnet and thereby vary the magnetic fields within the reaction well;

FIGS. 24A-24B are perspective, partially schematic views depicting a mechanically displaced electromagnet configured to move the bead in accordance with one aspect of the present invention which utilizes a directional switch of the current through the coils of the electromagnet in order to displace the electromagnet and thereby to vary the magnetic fields within the reaction well;

FIG. 25A is a top view depicting a mechanically displaced magnet being placed on a rotating shaft which is configured to rotate the magnet about the reaction well and thereby vary the magnetic fields within the reaction;

FIGS. 25B-25C are top views of the system shown in FIG. 25A;

FIG. 26 is a side, partially schematic view depicting the use of the electromagnet configuration of FIGS. 23A-23C as used in conjunction with an optics head;

FIG. 27 is a side, partially schematic view depicting the use of the rotating shaft configuration of FIGS. 25A-25C as used in conjunction with an optics head;

FIGS. 28A-28C are side, partially schematic views depict an alternative rotating shaft configuration which rotates magnets and their corresponding magnetic fields in and out of range of the reaction well in yet another embodiment of the present invention;

FIGS. 29A-29B are side, partially schematic views depicting the use of the rotating shaft configuration which rotates magnets and their corresponding magnetic fields in and out of range of the reaction well both above and below the reaction well; and

FIG. 30 depicts a flow chart embodying a method for achieving a homogeneous solution and reactants during a heated PCR application.

Reference will now be made to the exemplary embodiments illustrated, and specific language will be used herein to describe the same. It will nevertheless be understood that no limitation of the scope of the invention is thereby intended.

### DETAILED DESCRIPTION

Definitions

As used herein, the singular forms “a” and “the” can include plural referents unless the context clearly dictates otherwise. Thus, for example, reference to “a heating unit” can include one or more of such units.

As used herein, the term “substantially” refers to the complete or nearly complete extent or degree of an action, characteristic, property, state, structure, item, or result. As an arbitrary example, an object that is “substantially” enclosed is an article that is either completely enclosed or nearly completely enclosed. The exact allowable degree of deviation from absolute completeness may in some cases depend upon the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. As another arbitrary example, a composition that is “substantially free of” an ingredient or element may still actually contain such item so long as there is no measurable effect as a result thereof.

As used herein, the term “about” is used to provide flexibility to a numerical range endpoint by providing that a given value may be “a little above” or “a little below” the endpoint.

Relative directional terms are sometimes used herein to describe and claim various components of the present invention. Such terms include, without limitation, “upward,” “downward,” “horizontal,” “vertical,” etc. These terms are generally not intended to be limiting, but are used to most clearly describe and claim the various features of the invention. Where such terms must carry some limitation, they are intended to be limited to usage commonly known and understood by those of ordinary skill in the art. In particular, the term “side” is sometimes used herein to describe a boundary of a vessel or a well. It is to be understood that such term is not limited to a lateral portion of the vessel or well, but can include a top, bottom, lateral portion, etc.

As used herein, the terms “closed” or “sealed” reaction well or container are to be understood to refer to a well or container that is sealed on all sides (e.g., there is no “open” top or side portion). A closed or sealed well or container may be closed or sealed to varying degrees. In one aspect, the well or container is sealed so as to be liquid-tight: that is, liquid cannot enter or exit the well or container during normal operation. In one aspect, a closed or sealed well or container can be closed to the extent that mixing beads contained within the well or container cannot exit the container. In one aspect, the well or container can be gas-tight: that is, no gas can enter or exit the well or container during normal operation. It is to be understood that various fluid (gas or liquid) inlet or egress ports may be formed in or coupled to the vessel or container for the purpose of introducing matter into, or removing matter from, the vessel or container. However, such ports can be closed or sealed to create a closed or sealed well or vessel for the purposes of testing, as outlined herein. A vessel having such ports associated with it can still be considered a closed or sealed vessel, as those terms are used herein, so long as the vessel is closed or sealed during testing.

As used herein, a chemically inert or non-reactive coating or component is a coating or component that either does not chemically react with the solution within a vessel or container, or to the extent any chemical reaction does occurs, such reaction does not interfere with the test being conducted within the vessel (be that a PCR test or another test). In other words, a chemically inert or non-reactive coating or component is inert to the extent that the test being performed is not affected by the chemically inert or non-reactive coating or component.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually.

This same principle applies to ranges reciting only one numerical value as a minimum or a maximum. Furthermore, such an interpretation should apply regardless of the breadth of the range or the characteristics being described.

Invention

The polymerase chain reaction is an important tool for use as a precursor for a number of activities, such as the identification of small amounts of a particular genetic material in a sample, measurement of how much genetic material is present in a sample, or generation of enough genetic material for use in various applications.

Conventional thermocyclers have taken a number of forms. The most common thermocyclers utilize a plurality of sample vials placed into a large, solid, thermally conductive block. Each vial is manually loaded with sample DNA desired to be amplified, hereinafter sometimes referred to as “template DNA,” and the chemical constituents necessary for the polymerase chain reaction. The steps of the PCR process are performed in a laboratory by skilled technicians.

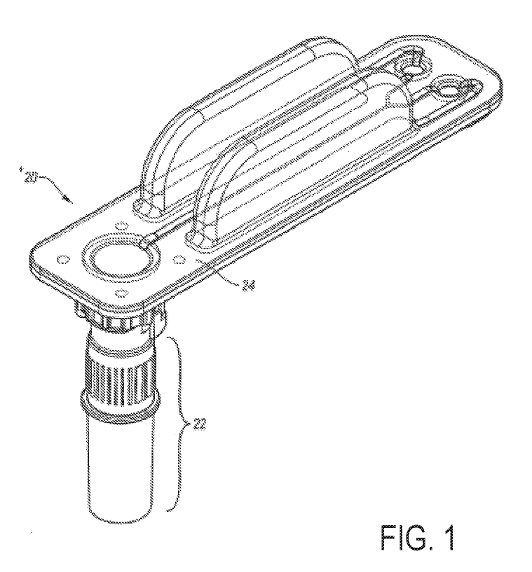

Referring to FIG. 1, the present invention provides a sample processing module 20 which simplifies the PCR process, various aspects of which are disclosed in the co-pending U.S. patent application Ser. No. 11/958,332, filed Dec. 17, 2007 and entitled “PCR Sample Processing Module,” which is incorporated in its entirety for all purposes by this reference. Sample processing module 20 comprises a sample assembly 22 and a PCR assembly 24. Sample assembly 22 is used for collection and optional processing of a biological sample, template DNA, or a template RNA. PCR assembly 24 is used for the performance of PCR amplification of template DNA.

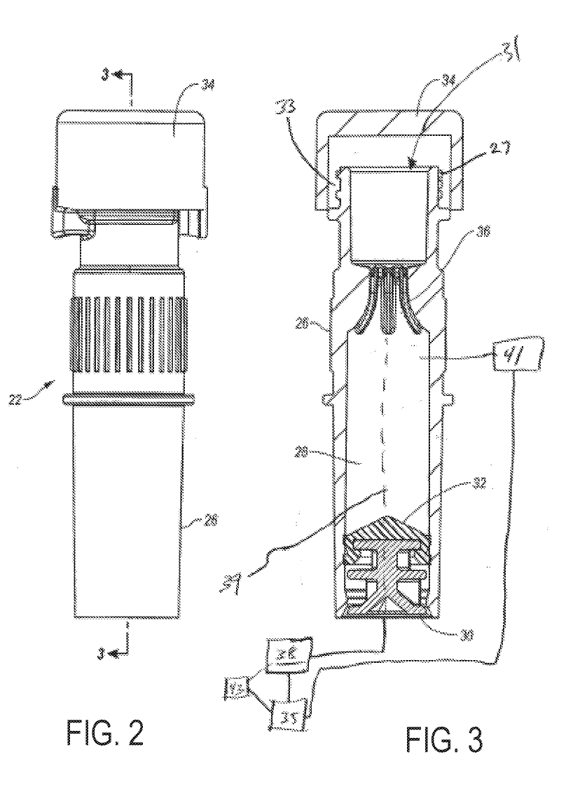

FIG. 2 is a side view of sample assembly 22 prior to being attached to PCR assembly 24. As more easily seen in the cross-sectional view of sample assembly 22 taken along section line 3-3 of FIG. 2, FIG. 3 shows sample assembly 22 as having a body 26 which contains an interior cylindrical cavity 28 with an open end 31 for receiving a sample of a biological material or a solution, such as a carrier fluid, containing template DNA or RNA. Sample assembly 22 is advantageously provided with a removable threaded cap 34 having cap threads 33 that mates with corresponding body threads 27 on the body 26 of the sample assembly 22 so as to protect cavity 28 from contamination prior to use and to secure the contents once a sample is placed within the cavity 28 but prior to the sample assembly 22 being secured to the PCR assembly 24. Of course, other ways of mating the body 26 to the cap 34 and the PCR assembly 24 can be used, such as friction fits, seals, clasps, latches, and the like, all fall within the scope of the disclosure.

The upper end of cavity 28 may be provided with an anti-splash structure 36. The bottom end of cavity 28 is sealed by a movable plug 30, which on its upper end is fitted with a seal 32 that prevents escape of the contents of the cavity. The movable plug 30 is mechanically coupled to a linear actuator 38 that moves the moveable plug 30 into and out of the cavity 28 along axis 39. In some embodiments, the linear actuator is actuated manually, such as by a user depressing a plunger (not shown). In other embodiments, the linear actuator 38 is connected to a controller 35 that generates a movement signal and transmits it to the linear actuator 38, that may include any of various types of electric motors, screw drives, and equivalent systems. The controller 35 can be a general-purpose computer with a program written to affect the described actions and/or it may be a specific instruction computer or chip. Optionally, the controller 35 stops sending the movement signal and/or transmits a stop signal after a given period of time, during which the distance traveled may be calculated for the rate at which a linear actuator 38 moves the moveable plug 30.

Optionally, a pressure sensor 41 operably coupled to the sample assembly 22 and/or the PCR assembly 24 (FIG. 8) is coupled to the controller 35. The pressure sensor 41 is configured to generate a pressure signal reflective of the pressure in the cavity 28 and/or one or more areas of the PCR assembly 24, such as the PCR reaction vial, well, chamber or vessel 56 (FIG. 8) and transmit the pressure signal to the controller 35. The controller 35, in response to a pressure signal, stops sending the movement signal and/or transmits a stop signal to the linear actuator 38. Optionally, a force gauge 43 configured to detect a force applied by the linear actuator 38 to the moveable plug 30 and to generate a force signal representative of the force is coupled to the controller 35. Embodiments of the force gauge include electrical, electro-mechanical, mechanical switches, and binary devices that change state when a selected or target force is met. The force gauge 43 transmits the force signal to the controller 35. The controller 35, in response to the force signal, stops sending the movement signal and/or transmits a stop signal to the linear actuator 38.

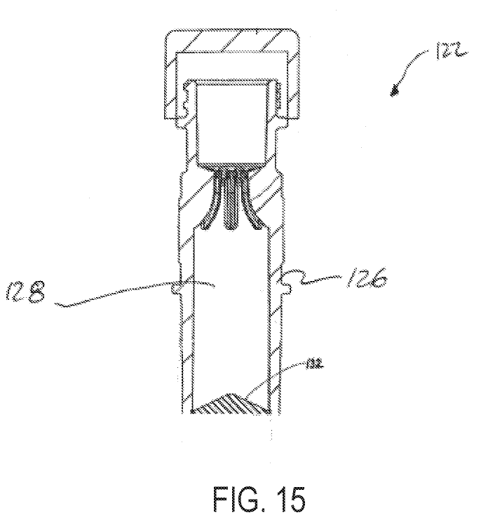

Rather than a moveable plug 30, another embodiment of sample assembly 122 illustrated in FIG. 15 includes a flexible membrane or bladder 132 incorporate or mechanically coupled to the body 126 of the sample assembly 122. The flexible membrane 132 acts similarly to the moveable plug 30 in that it is deflected inward into the cavity 128 of the sample assembly 122 and, in so doing, urges the sample into the PCR assembly 24 as described below. A possible advantage of using a flexible membrane 132 is that it may reduce the risk of a leak occurring at higher pressures at the interface where the flexible membrane 132 couples to the body 126 as compared to the seal provided by the moveable plug 30 against the body 26. The flexible membrane 132 may be deflected inward through the use of air pressure or other force applied to the flexible membrane 132 as an alternative to the linear actuator 38.

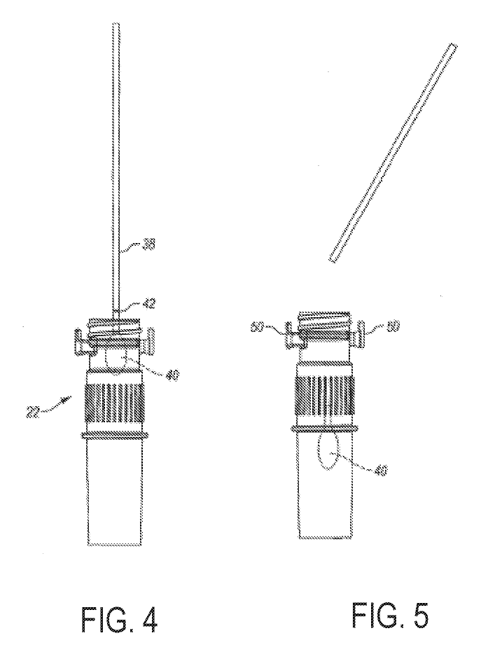

Sample assembly 22 may be preloaded with a solution or dried constituents, reagents, base chemicals, such as deoxyribonucleic triphosphate (dNTP), and the like, or may be provided empty until use. Although a template DNA may be placed into the sample assembly 22 for use with the PCR sample processing module 20, it is contemplated that the sample assembly 22 can utilize intact biological samples. FIGS. 4 and 5 depict the use of a swab 38 which has been used to collect a biological sample, for example from a patient's throat. Swab tip 40 may be separated from the swab 38 at a weakened area 42, allowing swab tip 40 to drop into cavity 28 of the sample assembly 22. The sample assembly 22 may then be subjected to an ultrasonic treatment to release DNA and RNA from the biological sample in accordance with the methods and apparatus of copending U.S. patent application Ser. No. 11/958,299, filed Dec. 17, 2007 and entitled “Ultrasonic Release of DNA and RNA,” the disclosure of which is incorporated herein by reference in its entirety for all purposes.

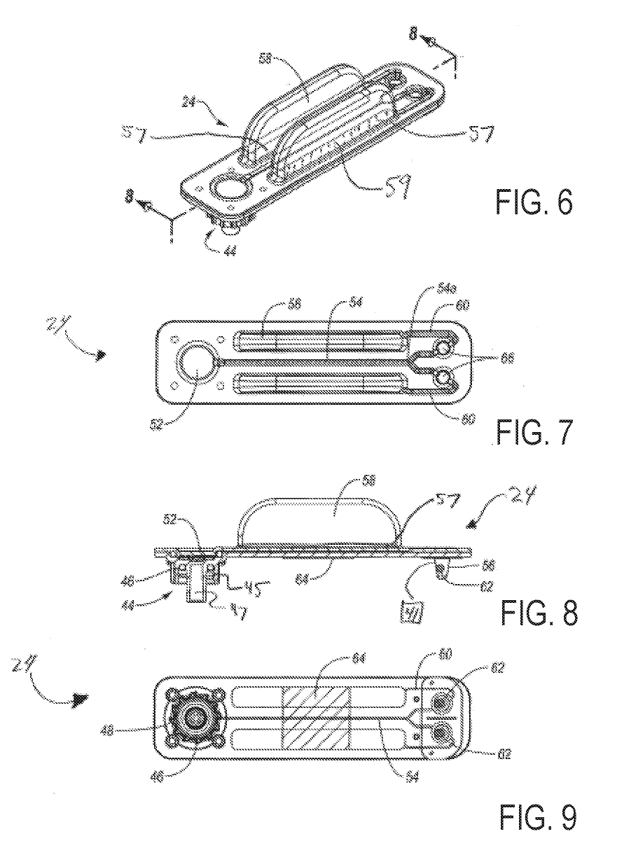

FIGS. 6-9 and 11-13 depict different views of the PCR assembly 24 of FIG. 1. PCR assembly 24 is provided with an interface 44 that mates with the open end 31 and the body threads 27 of the sample assembly 22 illustrated best in FIG. 3. As best seen in FIG. 8, a sealing element 46, such as an O-ring, rubber gasket, and the like, is provided to act as a seal between the PCR assembly 24 and sample assembly 22. It is preferred that interface 44 and the sample assembly 22 be provided with a locking structure 48, illustrated in FIG. 9, that prevents the sample assembly 22 from being removed once it has been secured to the PCR assembly 24 so as to protect against subsequent exposure of the contents of the sample assembly to users or handlers of the sample processing module 20, a feature that is particularly desirable when working with virulent substances.

The locking structure 48, for example, optionally includes ratchet notches that interface with flanges 50 on the sample assembly 22 (see FIG. 5) so as to allow sealing attachment of the sample assembly 22 to the PCR assembly 24, but to thereafter prevent removal of the sample assembly 22 from the PCR assembly 24. A fluid conduit 47 in the PCR assembly 24 provides a route through which fluids and the sample contained within the cavity 28 of the sample assembly 22 travel. A filter 52 is provided over the fluid conduit 47 to prevent solid matter from passing from the sample assembly 22 into a fluid delivery channel 54 of the PCR assembly 24. Stated differently, solution containing template DNA to be amplified contained within the cavity 28 of the sample assembly 22 is urged through the filter 52 and into the delivery channel 54 by raising the movable plug 30 upwardly into cavity 28 so as to eject the solution from the cavity 28 into the delivery channel 54. Delivery channel 54 carries sample solution from the sample assembly 22 to one or more PCR reaction vials or wells or chambers 56. The illustrated PCR assembly 24 shows the use of two PCR reaction vials 56, each of which is provided with sample via a split in the delivery channel 54 shown at reference numeral 54a.

It is preferred that the sample processing module 20 be permanently secured in place following attachment of the sample assembly 22 to the PCR assembly 24 so as to protect against release of potentially hazardous materials. Inasmuch as this results in an enclosed, non-vented space which decreases in overall volume as the movable plug is advanced within cavity 28, it is preferred to provide one or more air chambers that serve to accommodate the reduction of volume without impeding flow of solution from the sample assembly 22 to the PCR reaction vials 45, and which also serve as a vent location for bubbles forming within the PCR reaction vials 56. The illustrated embodiment utilizes vent chambers 58 for this purpose, which communicate with PCR reaction vials 56 through vent channels 60 but which are otherwise sealed. Although FIGS. 6-9 show use of a separate vent chamber 58 for each of the PCR reaction vials 56, it should be understood that this is not required and that other configurations could provide the same functionality as vent chambers 58. For example, and as will be discussed below, embodiments of the PCR assembly 124 illustrated in FIG. 14 optionally forego a vent chamber altogether and provide a structure through which gases might be vented directly to the atmosphere.

It is preferred that each PCR reaction vial 56 contain a lyophilized bead 62 comprising the various constituents, hereinafter “the PCR reaction mixture,” required to amplify the template DNA supplied from the sample assembly 22. The PCR reaction mixture within the lyophilized bead 62 will include the primers necessary to amplify the template DNA, the polymerase, dNTP, and any other necessary constituents. More than one lyophilized bead 62 may be provided if that is more convenient or if various constituents of the PCR reaction mixture need to be isolated from one another prior to use.

The PCR reaction mixture will differ depending on the template DNA to be amplified. Inasmuch as PCR assembly 24 is provided in a preloaded form factor, a label should be attached which identifies the preloaded PCR assembly. FIGS. 8 and 9 show the use of a radio frequency identification (RFID) label 64, which can also be used to provide information to a thermocycler with which the sample module is used. Optimal operating conditions and protocols, such as those used by the controller 35 may differ depending upon the template DNA or the constituents of the PCR reaction mixture, so the RFID identification information can also be used to select a thermocycler program, which may include instructions regarding parameters such as target temperatures and cycle times, that are optimal for the contents of the PCR reaction mixture. It is preferred that PCR be monitored and controlled on a real-time basis.

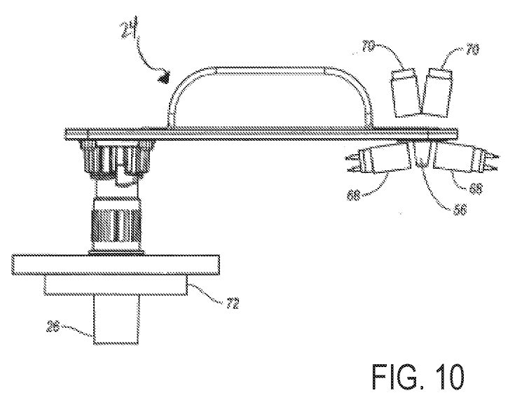

FIG. 7 depicts the provision of lenses 66 to assist in direct visual observation of the contents of PCR reaction vials 56. FIG. 10 depicts the use of optical excitation sources 68 for use in the excitation of fluorescent constituents of the PCR reaction mixture, and photo receivers 70 which monitor fluorescent emissions through lenses 66. Copending U.S. Patent Application Publication Application No. US 2006/0152727, entitled “Fluorescence Detection System,” incorporated in its entirety by this reference for all purposes, contains additional details of fluorescent detection systems that could be implemented for use with PCR assembly 24.

Copending U.S. Patent Application Publication No. US 2006/018889, entitled “Methods and Apparatus for Controlling DNA Amplification,” incorporated in its entirety by this reference for all purposes, provides information regarding the control of PCR using real time information from an optical detection system.

As noted, bubbles may form with the sample and the carrier fluid during the filling of the reaction vials 56 and/or during the thermocycling process, particularly if the vapor pressure of the sample and carrier fluid is close to the temperature at which the denaturing process occurs, thereby raising the risk of unintentionally boiling the sample. For example, an ideal denaturing temperature for a typically is from about 94 degrees centigrade to about 96 degrees centigrade. However, the boiling point of water at a city at the altitude of Denver, Colo., for example, is at about 97 degrees centigrade. Thus, to avoid unintentionally boiling the sample during a PCR reaction occurring in Denver requires the use of very accurate and, consequently, expensive thermal control system. In addition, bubbles of dissolved gases might come out of solution during the thermocycling process as the capacity of the carrier fluid to maintain the dissolved gases in solution decreases during the heating phase of the thermocycle. Further, the presence and development of bubbles within the fluid alters the volume of the fluid. This occurs as the volume of the bubbles change much more significantly than the volume of the fluid and sample change during thermocycling. The change in the volume of the bubbles cause the fluid and entrained DNA sample to flow in and out of the reaction vessel. Such flows of the fluid in and out of the reaction vessel may alter and/or dilute the concentration of the DNA sample and/or the PCR reaction agents with in the reaction vessel.

These bubbles pose several potential difficulties in accurately replicating and analyzing samples that undergo a PCR reaction. For one, it has been discovered that bubbles often form under lenses 66, and these bubbles can result in inaccurate readings by photo receivers 70.

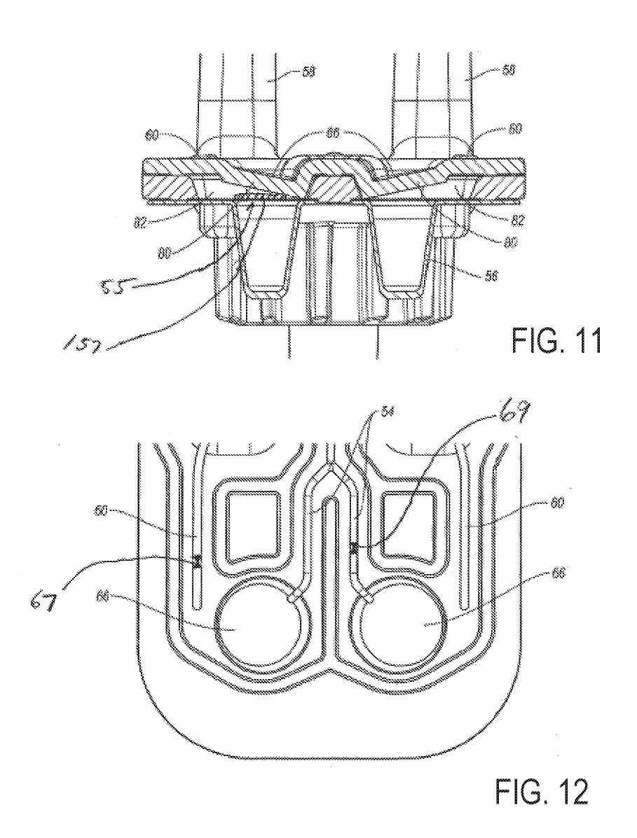

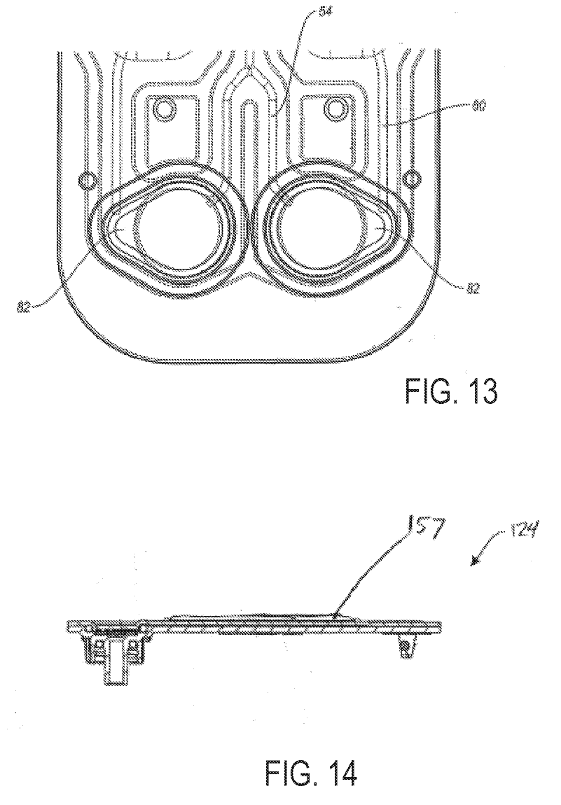

To manage and mitigate the effect any bubbles might have after the bubbles have developed, FIGS. 11-13 depict a configuration of lenses 66 and associated structure which is useful to direct bubbles away from the lenses so that the accuracy of readings is not impeded. FIG. 11 shows the use of an angled portion 80 formed in the structure of the PCR assembly overlying reaction vials 56. This angled portion 80 serves to direct bubbles away from the area underlying the lenses 66, and toward connecting vent channels 60 which lead to vent chambers 58. As best seen by reference to both FIGS. 11 and 13, using a teardrop shaped cavity 82 further assists in directing bubbles toward vent channels 60.

While the angled portion 80 acts to shepherd and guide bubbles away from the lenses 66 after the bubbles form, it is desirable to reduce the number of bubbles generated, in the first instance. Further, it is desirable to reduce the volume of any individual bubble after the bubble has formed. In so doing, the efficacy of the angled portion 80 to shepherd or guide bubbles that are created could possibly be improved.

Applicants have discovered that including an optional flow restriction device 57 (FIGS. 6 and 8) downstream of the reaction vials 56 and between the reaction vials 56 and the vent chambers 58. In one embodiment, the flow restriction device 57 in FIGS. 6 and 8 is a permeable membrane that is sealed, such as by welding by heat, laser, sonically, and the like, to the PCR assembly 24. In the embodiment illustrated in FIGS. 6 and 8, each permeable membrane 57 is substantially the same length and width of the associated vent chamber 58. Of course, it will be understood that the dimensions of the permeable membrane 57 may be selected as appropriate. In addition, the location of the permeable membrane can be located in different locations, including within an inner diameter of the vent channel 60.

In another embodiment, the permeable membrane 57 may form at least a portion of a wall 55 of the reaction vial 56, as illustrated in FIG. 11. An advantage of the embodiment in which the permeable membrane 157 forms a portion of a wall 55 is that it may prevent PCR reagents, the sample, and/or the carrier fluid from being flushed or diluted out of the reaction vials 56 during the filling and/or pressurization process. Further, any bubbles that do come out of solution may pass through the permeable membrane and into the vent channel 60.

An attribute of embodiments of the permeable membrane 57 is that it is selectively permeable. That is, the permeable membrane 57 is permeable to selected fluids, such as water vapor and formerly dissolved gases that come out of solution of the sample and carrier fluid. At the same time, the permeable membrane resists and/or prevents the flow of other fluids, such as water, the carrier fluid, and other liquids from passing through the permeable membrane 57. As a result, gases can flow through the permeable membrane 57 and into the vent chamber 58 while the sample and carrier fluid are retained within the reaction vial 56 and, depending upon the location of the permeable membrane 57, the vent channel 60.

Optionally, the membrane 57 and other embodiments thereof, include a framework or structure 59 to minimize or reduce any bulging or deflection of the permeable membrane 57 when a pressure differential exists between the two sides of the permeable membrane 57, as illustrated in FIG. 6. A deflection of the permeable membrane 57, if unchecked, may cause a distortion or change in the relative volumes of the vent chamber 58 and the volume upstream of the vent chamber 58 (i.e., the reaction vials 56 and associated fluid channels). That is, as the permeable membrane 57 bulges more of the carrier fluid and sample flows through the reaction vial 56, possibly causing more of any PCR reagents being flushed out of the reaction vials 56 and into the vent channel 60. Such a change in the relative volume could require a higher or lower fluid pressure and, consequently, more or less fluid than anticipated to obtain a selected pressure. Thus, a framework or supporting structure 59, such as ribs or a lattice, optionally is inserted within the permeable membrane or on one or both sides of the permeable membrane 57. The supporting structure 59 resists the deflection of the permeable membrane 57 when it is subject to a pressure differential between its two sides. In some embodiments, rather than a physical structure 59 the permeable membrane 57 optionally includes a matrix of load supporting fibers and other similar enhancements that improve the resilience and stiffness of the permeable membrane.

Another embodiment of a flow restriction device is a valve 67 located within the vent channel 60 and, optionally, valve 69 located within fluid delivery channel 54, illustrated in FIG. 12. The valve may be selectively operated by a user or under command from a controller 35 to open partially and/or fully and to selectively close the valve. Closing the valve 67 after any gases have been vented to the vent chamber 58 allows a pressure above ambient to be applied to the sample within the reaction vial 56 as will be described in further detail below. An embodiment of the valve includes those made from elastomeric elements. The flow restriction device allows a pressure to be applied to the sample and carrier fluid contained within the reaction vial 56. That is, the pressure within the reaction vial 56 can be maintained above ambient pressure, such as from about atmospheric/ambient pressure to about 80 pounds per square inch (psi) above ambient pressure and, in some embodiments, pressures even higher. In other embodiments, the flow restriction device provides the capability of maintaining the pressure within the reaction vial from about 15 psi to about 60 psi above ambient pressure.

In the embodiment of the flow restriction device that comprises the permeable membrane 57, pressure is provided in the following way. As noted, the moveable plug 30 is moved linearly, urging the sample and entrained carrier fluid to flow through the fluid delivery channel 54, into the reaction vials 56, through the vent channel 60 until it reaches the permeable membrane 57. Gases, air, and other bubbles within the sample and carrier fluid will pass through the permeable membrane, while the sample and carrier fluid presses against the permeable membrane 57. Pressure above ambient can be created within the reaction vials 56 by causing the moveable plug 30 to further advance. As discussed above, the movement of the moveable plug 30 can be controlled by the controller in response to a force signal or a pressure signal, whether at the sample assembly 22 and/or the reaction vial 56. Stated differently, pressurizing the fluid against the permeable membrane 57 serves as a method to detect when a selected volume or amount of the sample and carrier fluid has entered the reaction vial 56 because any gases would have permeated the permeable membrane 57 and entered the vent chamber 58. When this occurs and the fluid reaches the permeable membrane 57 and fails to pass through the permeable membrane, a detectable increase in the pressure occurs quickly. In some instances, the pressure increase might be quite abrupt, thus indicating that the PCR assembly 24 is adequately filled to perform the PCR reaction. Thus, the rise in pressure serves, in part, as a method to detect when the sample and carrier fluid has reached the permeable membrane 57.

Another benefit of pressurizing the sample and carrier fluid against the permeable membrane 57 is that the stability of the pressure and/or force measured serves as a method of detecting leaks. That is, if the pressure and/or force measured at the moveable plug 30 were to decrease would be an indicator suggestive of a leak allowing fluid to escape from at least one of the sample assembly 22 and PCR assembly 24. Such an indication would permit a user to investigate the cause, run a new sample, and/or take other supplemental measures, particularly if the sample to be tested is hazardous.

In addition, the volume of the vent chambers 58 may be adjusted in part, to obtain a desired pressure within the reaction vial 56 and the pressure created within the vent chamber 58 by any gas that migrates into the vent chamber 58. That is, in some embodiments, the volume of the vent chamber 58 is a function of the dimensions of the cavity 28 of the sample assembly 22 and the reaction vial 56, and a desired or selected pressure to be obtained in the vent chamber 58 and the reaction vial 56, as well as the type of sample and carrier fluid as well as the reaction to occur within the reaction vessel. By calibrating the volumes of the vent chamber 58, the reaction vials 56 and the pressures within each when in use, the pressure differential across the permeable membrane 57 can be managed and optimized to improve the efficacy of the permeable membrane 57 and to ensure that the pressure differential does not exceed the design limits of the permeable membrane 57.

In some embodiments, the volume of the vent chamber 58 is sufficiently small such that the entry of gas into the vent chamber 58, as discussed above, creates sufficient back pressure so as to eliminate the need for a flow restriction device, such as the permeable membrane 57 or valve 67. In such an embodiment, the design of the vent chamber 58 is such that vent channel 60 turns upward and exits into the vent chamber 58, creating a u-tube hydrostatic effect. The u-tube prevents the gases from migrating back into the reaction vial 56 after they reach the vent chamber 58.

Applying a pressure above ambient to the sample and carrier fluid within the reaction vial 56 provides several benefits. For example, applying a pressure to the sample in the reaction vial 56 increases the boiling temperature of the sample and the carrier fluid. Thus, in the example previously described for Denver, Colo., the unpressurized sample boils at about 97 degrees centigrade, quite close to a selected denaturing temperature of from about 94 degrees centigrade to about 96 degrees centigrade. The sample under pressure, however, boils at a temperature higher and, depending on the pressure, sometimes significantly higher than 97 degrees centigrade. That is, because the boiling temperature is raised significantly away from a desired denaturing temperature, it is possible to forego very accurate and, consequently, very expensive temperature control methods. In so doing, bubbles that might otherwise be inadvertently created by boiling or coming out of solution are not created and, therefore, avoid the issues bubbles pose for optical scanning systems as discussed above.

Another advantage of creating a pressurizable cartridge as disclosed is that higher pressure at which the sample and the carrier fluid is maintained reduces the size of bubbles that are present and reduces the change in the volume of those bubbles as the thermocycling process occurs. As noted above, the change in the volume of the bubbles causes a pumping action by which the sample and carrier fluid may move into and out of the reaction vial by the changing volume of the bubbles. Thus, using pressure to minimize the volume of the bubbles reduces this pumping action that might cause dilution of PCR reagents in the reaction vial 56. In addition, reducing the volume of the bubbles reduces the effect those bubbles have on the optical scanning systems. That is, smaller bubbles will cause less noise in the optical signal used with fluorescence detection systems, as discussed above.

Yet another advantage is that the reaction vials 56 optionally are formed of a thin plastic and shaped with a taper to ensure good contact with the heating/cooling block that is used to heat and cool the reaction vial 56 and the sample therein during the thermocycle process. Holding the sample and carrier fluid at a higher pressure within the reaction vial 56 causes the thin plastic wall 55 (FIG. 11) of the reaction vial 56 to bulge slightly and press more tightly against the heating and cooling block, thereby improving the heat transfer thereto and the efficiency of the thermocycle process.

FIG. 10 depicts the optional use of a sonic transducer 72 in association with the sample assembly. Copending U.S. patent application Ser. No. 11/958,299, filed Dec. 17, 2007 and entitled “Ultrasonic Release of DNA and RNA” is incorporated in its entirety for all purposes by this reference, and discloses methods and apparatus for releasing DNA or RNA from biological materials using sonic energy.

As has been noted, the PCR process operates on DNA. When it is necessary to detect RNA rather than DNA, the RNA must first converted to DNA before PCR can be utilized.

Co-owned U.S. patent application Ser. No. 11/733,035, filed Apr. 9, 2007 and entitled “Rapid Reverse Transcription of PCR,” and incorporated by reference in its entirely herein, discloses methods and apparatus for use in forming template cDNA from template RNA, and further discloses incorporating the appropriate constituents for this process into a PCR reaction mixture and performing the transcription step prior to the PCR.

Any suitable thermocycler may be used to bring the sample and PCR reaction mixture to the desired PCR target temperatures, but it is currently preferred to use a thermocycler of the type disclosed in copending U.S. patent application Ser. No. 11/697,917, filed concurrently herewith and entitled “Rapid Thermocycler, and which application is incorporated by reference in its entirely herein.

Turning now to further embodiments of the technology in which mixing apparatuses and methods are utilized in combination with the pressurizable cartridges discussed above, it has been recognized that in order for chemical reactions or biochemical reactions to be efficient the solution of reagents must be as homogeneous as possible. In the case of Polymerase Chain Reactions (PCR) the reagents, enzymes, primers, probes, target templates, etc., in solution need to be as homogeneous as possible so that efficient amplification of the target can occur. Many reactions also require a uniform temperature throughout the solution in the reaction well for the reaction to be efficient. PCR also requires uniform temperatures at denature, annealing and reverse transcription for efficient amplification of the target DNA segment to occur.

Mixing the solution of reagents prior to starting the reactions and in the case of PCR amplification, will often satisfy the requirement of homogeneity and in an open system it is usually done as the reagents are added to the reaction well. The mixing step for homogeneity within a closed cartridge system becomes much more difficult. Where uniform temperature is required, either the solution in the reaction well needs to have its temperature tightly controlled, or the solution needs to be mixed so that temperature gradients within the solution are minimized.

The present technology addresses these issues in a variety of manners. In one embodiment, a method of mixing chemical reagents or biochemical reagents (such as PCR reagents in a reaction well or mixing chamber) is provided. The method can be accomplished in a standalone well or chamber or within a closed cartridge (e.g., container) system. The method can include using beads that are made from magnetically responsive materials or alloys and coated with a chemically or biochemically inert or non-reactive coating such as parylene. The method includes various means or manners to move the beads inside the reaction well or mixing chamber, thus causing mixing to occur.

In one aspect of the invention, beads made of magnetically responsive material are coated with a material that is inert to chemical or biochemical reactions. These beads can be used to mix the chemical or biochemical solution to provide homogeneity and reduce the effects of any thermal gradients within the mixing chamber or reaction well.

In another aspect of the invention, various means or methods are carried out to move the beads within the mixing chamber or reaction well. The present technology can cause sufficient mixing to achieve the desired homogeneity and reduction of thermal gradients, thus enhancing the efficiency of the desired reaction.

The present invention provides a convenient, compact, effective and inexpensive solution to the problems presented by conventional mixing means. In one embodiment, only one actuating magnet is required to achieve mixing and the actuating magnet is remote from the immediate vicinity of the reaction well. As such, vibration levels are intrinsically low and are easily controlled. As the actuation system is non-invasive, sealed reaction vessels pose no limitation. The active mixing means can be controllably positioned well away from the optical paths required to monitor the reaction. In some embodiments, the system can directly verify that mixing motion is occurring while the reaction progresses.

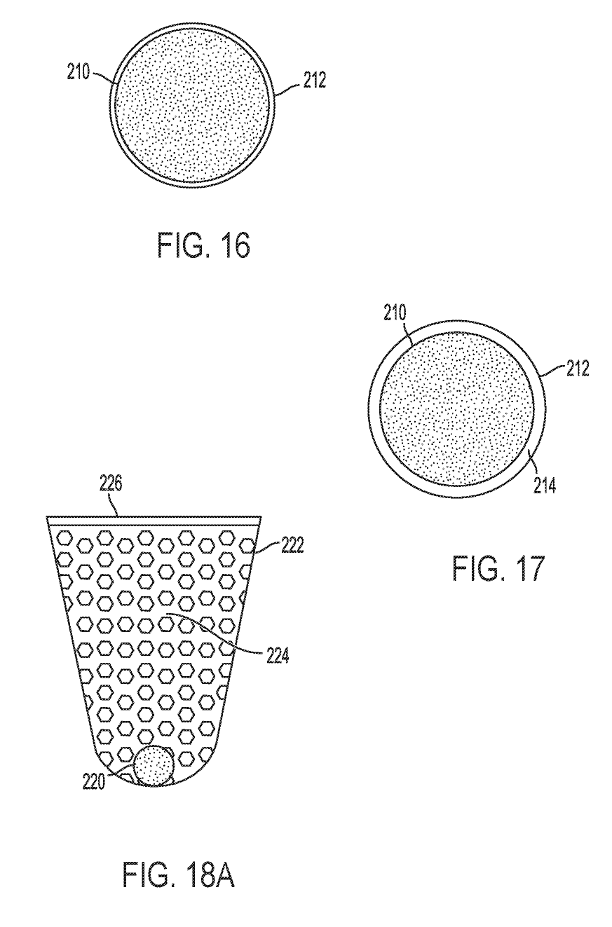

An embodiment of the invention is illustrated generally in FIG. 16. In this aspect, the bead 210 can be made of a magnetically responsive or ferromagnetic material such as iron, nickel, cobalt or some alloy thereof. While the bead can be magnetized, in many embodiments it is not magnetized. The bead 210 can be coated with a thin chemically inert coating 212. The bead can be formed from a variety of materials, and can have a variety of coatings (or no coating at all). The bead can include a homogenous or nonhomogeneous construction. That is, it can be formed of a single material, or multiple materials combined or mixed together.

The bead 210 can be sized according to the needs of the mixing chamber and the strength of the magnet used to move the bead. In one preferred embodiment, the bead 210 is steel shot that is about 1.5 to about 1.85 mm in diameter and the coating 212 is about 5 microns of parylene. In this embodiment, the mixing chamber or vessel has a volume of about 50 μL and includes a generally conic shape, terminating in a generally rounded bottom, as shown in the various figures.

Another embodiment of the invention is shown in FIG. 17. Once again, the bead 210 is made of a magnetically responsive material such as iron, nickel, cobalt or some alloy thereof, but it is not a magnet nor has it been magnetized. The bead 210 is coated first with a thin optical coating 214 to counteract any negative optical effect that the natural color of the bead might have on any optical detection system used to read the progress of the chemical or biochemical reaction in the mixing chamber. The thin optical coating 214 can be white, such as titanium dioxide or a mirror type of coating such as nickel. The bead is then coated with a thin coat 212 of a chemically inert material such as parylene. Once again, the bead 210 should be sized according to the needs of the mixing chamber and the strength of the magnet used to move the bead, only in this case the extra layer of coating material is taken into consideration.

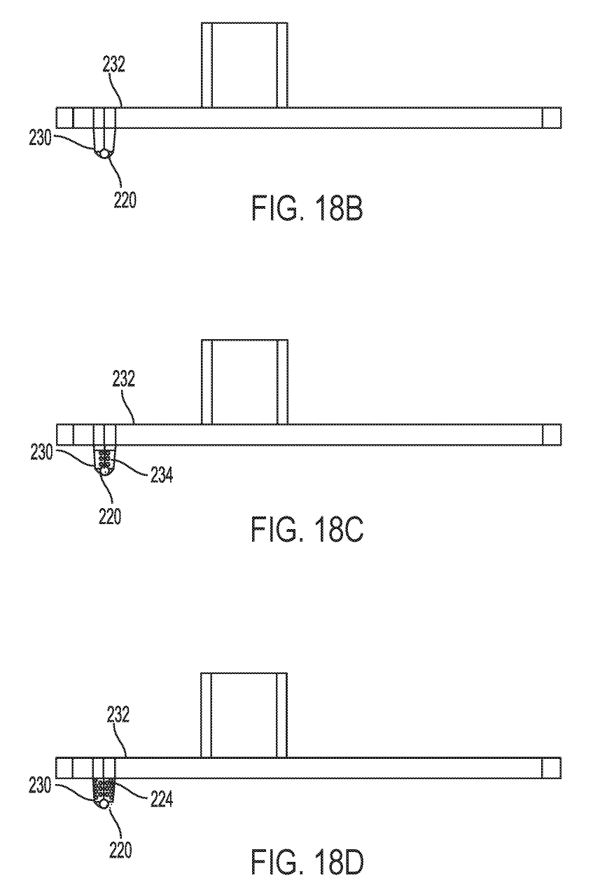

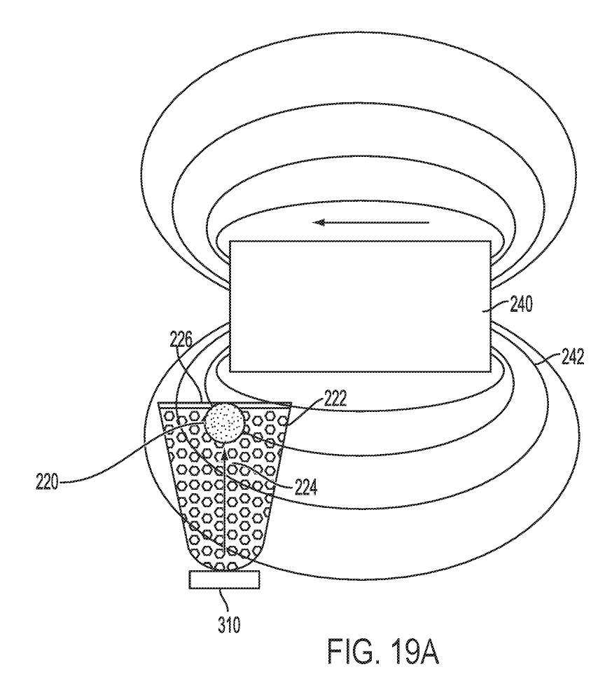

FIG. 18A shows a coated bead 220 as described in FIG. 16 placed inside a closed cartridge reaction well 222 that is also filled with a solution and various reagents 224. In the case of PCR, there can also be templates, probes, primers, etc., present. The well can include a barrier 226 that stops the bead's upward motion. The barrier is typically made of a material that does not shield the bead from magnetic flux. In the case that the progress of the reaction is monitored from above by an optics system, the barrier material and configuration should also accommodate the optics system. The barrier is essentially a lid or covering on a container within the well, or the well itself, that creates a closed vessel in which the various materials are held. The barrier can be formed of a variety of materials and can be attached to the vessel or reaction well in a variety of manners. The barrier can be removably attached to the vessel or well. Non-limiting examples include a “snap-on” attachment, threaded attachment, hinged attachment, and the like. In some cases, a pressure- and/or heat-sensitive film or material can be applied to create the barrier.

As the system provides suitable agitation of the solution with the mixing bead and magnet system, it does not require access to the solution with an external, invasive device, such as a mixing bar, stir stick or the like. In this manner, the sealed or closed vessel technology utilized herein avoids many of the disadvantages found with conventional systems. The present technology provides improved durability, reliability and accuracy over conventional system due to its compact and minimalistic design.

FIGS. 18B through 18D are examples of the coated bead 220 as described in FIG. 16 in the reaction chamber of a closed cartridge test system as the cartridge is being manufactured. FIG. 18B shows the bead 220 in the reaction well 230 of a closed cartridge or vessel 232. FIG. 18C shows the bead 220 included in the well 230 of a closed cartridge system 232 with lyophilized chemical or biochemical reagents, and in the case of PCR, with primers and probes 234. FIG. 18D shows the bead 220 included in the well 230 of a closed cartridge system 232 with a solution of chemical or biochemical reagents, and in the case of PCR, probes, primers, templates, etc.

Generally speaking, to move the bead and cause mixing to occur, a magnetic flux is brought into proximity of the reaction well or the mixing chamber containing the bead. The bead, being made of magnetically responsive material, will be drawn toward the magnetic flux and pass through the solution. The magnetic flux can be brought into the proximity of the well and the magnetically responsive bead by moving a permanent magnet into the appropriate position or energizing an electromagnet that is already in the appropriate position. Depending on the orientation of the mixing chamber or reaction well and the desired speed of mixing, either gravity or another magnetic flux can be used to draw the bead in the opposite direction from which it was first drawn. This back and forth or up and down action of the bead, done repetitively and at a fast-enough rate, will cause the components of the solution to mix.

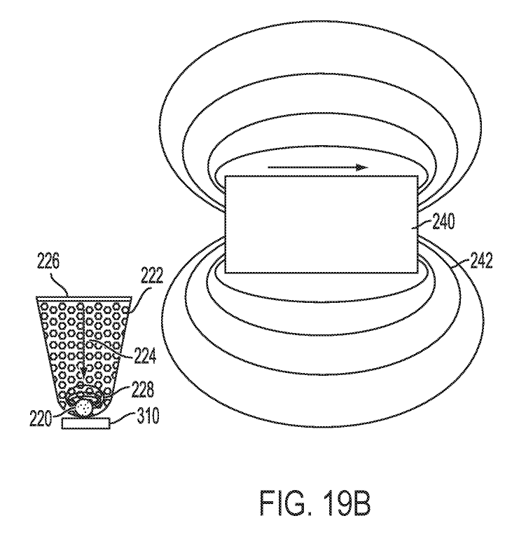

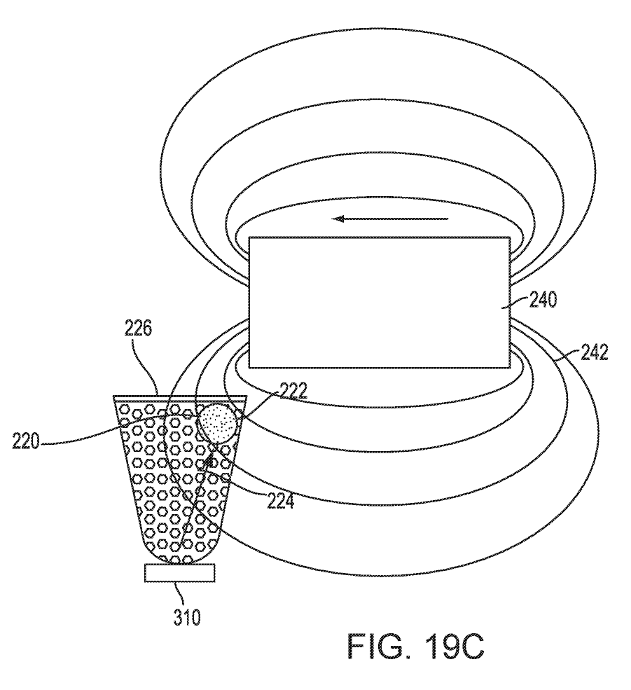

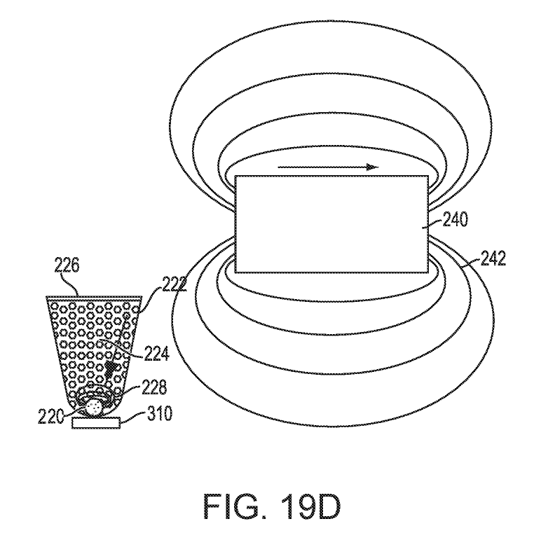

As a non-limiting example, FIGS. 19A-B show a magnet 240, which can be a rare earth magnet. In FIG. 19A, the magnet is being brought into position over a reaction well 222 containing reagents 224. In this manner, the magnetic flux 242 extends downwardly into the well 222 far enough to draw the coated steel bead 220 up to the barrier 226 of the reaction well 222. FIG. 19B shows that the magnet 240 is pulled far enough away from the reaction well 222 such that the magnetic flux 242 will no longer draw the bead 220 toward the magnet 240. At this point, the bead 220 will drop to the bottom of the reaction well 222. When relying on gravity to move the bead 220 to the bottom of the well 222 the magnet 240 must be drawn far enough away from the well 222 and the bead 220 that the magnetic flux 242 of the magnet 240 will not intersect with the temporary magnetic field 228 sufficient enough to move the bead 220 that is generated by the magnetic responsive bead.

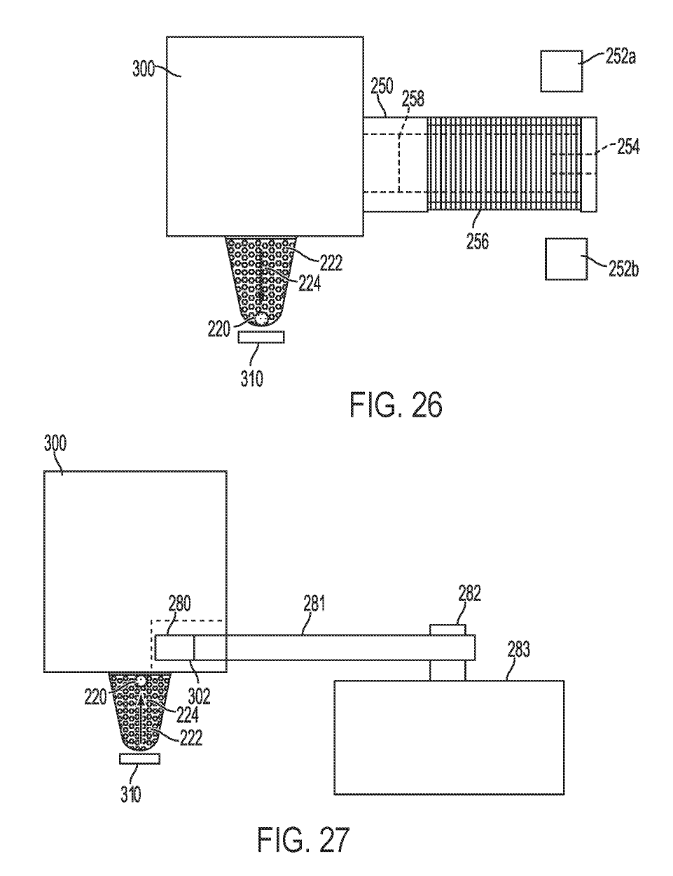

FIGS. 19C and 19D illustrate an example of the technology in which the magnet 240 is used to draw the bead 220 upward, and laterally sideward, within the well 222 without interfering with an optics system (not shown in detail here) that is directed downwardly into the well. As discussed above, the cover 226 of the well can be formed such that an optics head (300 in FIG. 26, as one non-limiting example) can be directed (e.g., sighted) downwardly into the well to detect various readings during a reaction. In the embodiment shown in FIG. 19C, the magnetic flux 42 can be generated by the magnet, in sufficient magnitude to cause the bead 220 to rise within the well, without the magnet optically obstructing the top portion of the well. In FIG. 19D, the magnet is moved to the right, which decreases the magnetic force on the bead such the bead moves downwardly within (and laterally toward a bottom center of) the reaction well.

These examples also illustrate another advantage of the technology. As the optics system can be directed downwardly into the reaction well, an optical viewing zone is effectively created in which various reactions can be detected by the optics system. As the bead is actuated by the magnetic system discussed, the bead can move into and out of this optical viewing zone. In the event the bead in some way interferes with the readings required for the test, the bead is intermittently moved away from any such interference, clearing the way for an unobstructed reading by the optics system. In addition, the system can use the presence or absence of the bead within the optical viewing zone to verify whether or not the bead is being properly moved through the solution within the reaction well. The optics system can be configured to monitor a position of the bead, either periodically or in real time, for various purposes.

Heat can be applied to the closed cartridge reaction well by heat source 310. It should be appreciated that heat source 310 may be any suitable heat source as recognized by one of ordinary skill in the art. In one specific example, a conventional cartridge heater is used. In this case, nichrome wire heating coils are inserted in holes formed in ceramic tubes. Pure magnesium oxide filler is vibrated into the holes housing the heating coils to allow maximum heat transfer to the stainless steel sheath. The heater then has a heliarc welded end cap inserted on the bottom of the heater and insulated leads are installed. While the heat source is shown near the bottom of the vessel or well, it is to be understood that it can be positioned in a variety of locations: aside, above, circumventing the vessel or well, etc. In addition, while the teachings herein refer to the heat source specifically, it is to be understood that thermal management of the contents of the well or vessel can be carried out using a cooling unit as well. Such a cooling unit can be positioned as discussed with the heating source, as would be appreciated by one of ordinary skill in the art.

As previously stated, the mixing motion of the bead in the configuration demonstrated in FIGS. 19A and 19B relies on gravity to pull the bead to the bottom of the well. This can be a limiting factor when it comes to the speed of the mixing action.

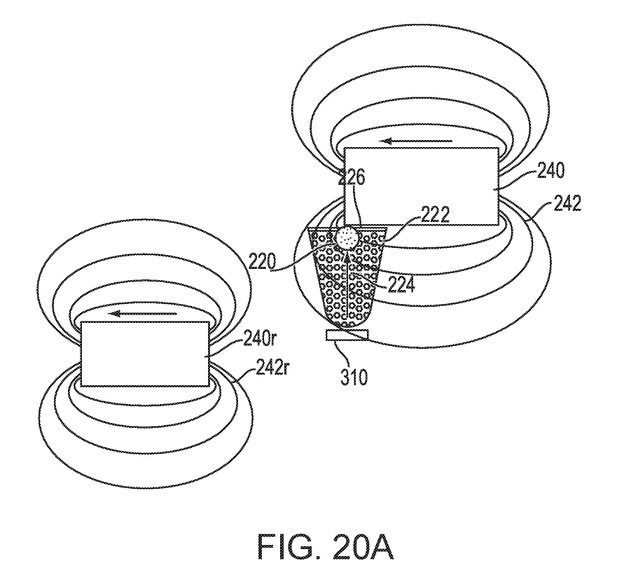

FIGS. 20A and 20B show an example of an embodiment that can greatly enhance the speed of the mixing. The bead 220 will be influenced by two magnetic fields 242 and 242r, each pulling the bead in the opposite direction from the other. In FIG. 20A, as in FIG. 19A, a magnet is brought into position over the reaction well 222 such that the magnetic flux 242 of the magnet 240 will draw the bead 220 to the top of the well 222 against the barrier 226. Next, as seen in FIG. 20B, the magnet 240 is pulled away from the well 222 so that its magnetic flux 242 no longer affects the bead 220. At substantially the same time, a magnet 240r near the base of the well 222 is brought into position under the reaction well 222 such that the magnetic flux 242r of magnet 240r draws the bead 220 towards the bottom of the well 222. This embodiment allows mixing to occur at a pace dependent on the depth of the well 222 and the speed at which the magnets 240, 240r can be moved. This dual magnet configuration increases the relative oscillating speed of the bead thus increasing the ability to maintain the homogeneity of the solution while heat is being applied via heat source 310.

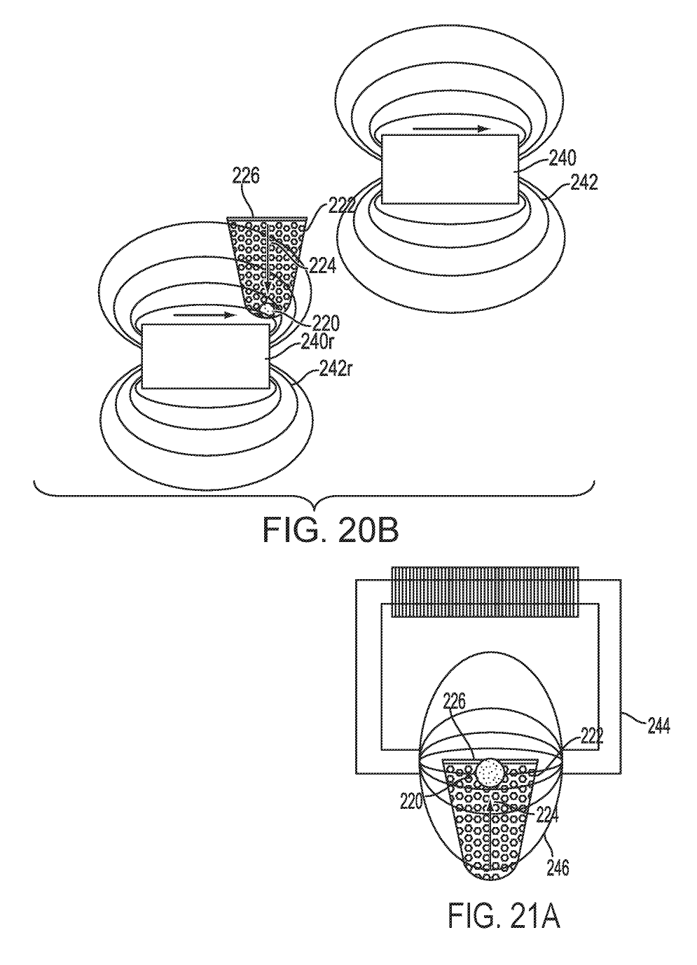

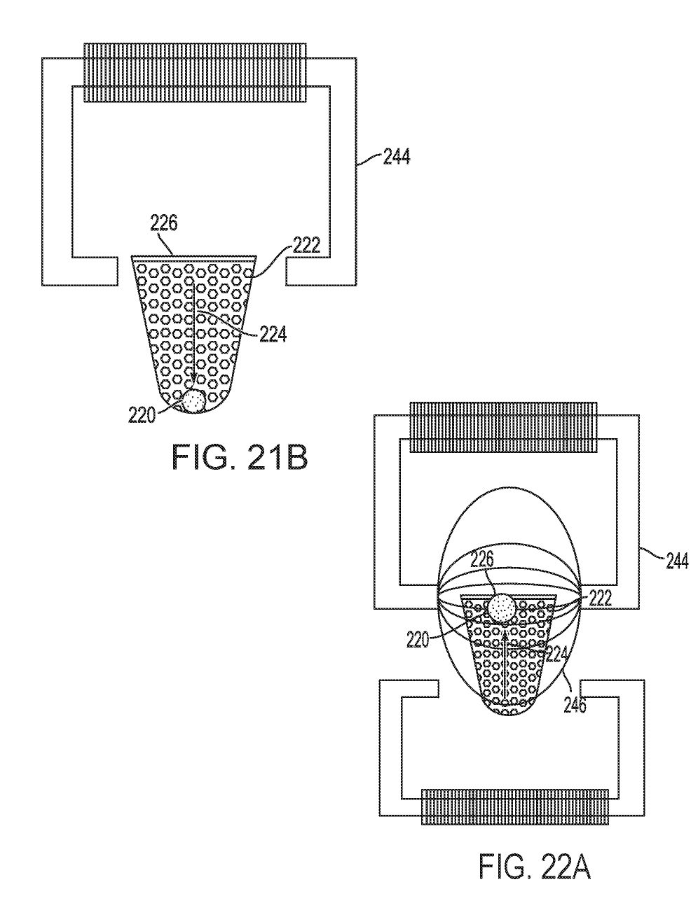

FIGS. 21A and 21B show an embodiment using an electromagnet 244 with a ‘C’ shaped core to bring a magnetic flux 246 into position to draw the bead 220 toward it and, in this embodiment, to the top of the well 222 and against the barrier 226. In FIG. 21A the electromagnet 244 is energized with a DC current adequate to generate enough magnetic flux 246 to reach into the well 222 and draw the bead 220 up through the solution 224. In FIG. 21B the DC current is turned off, causing the magnetic flux 246 to collapse, thus allowing the bead 220 to drop through the solution 224 to the bottom of the well 222. As in the case of using a magnet as described above and in FIGS. 19A and 19B, using gravity to return the bead 220 to its starting position limits the pace at which the bead 220 can be moved and the rate at which mixing can occur.

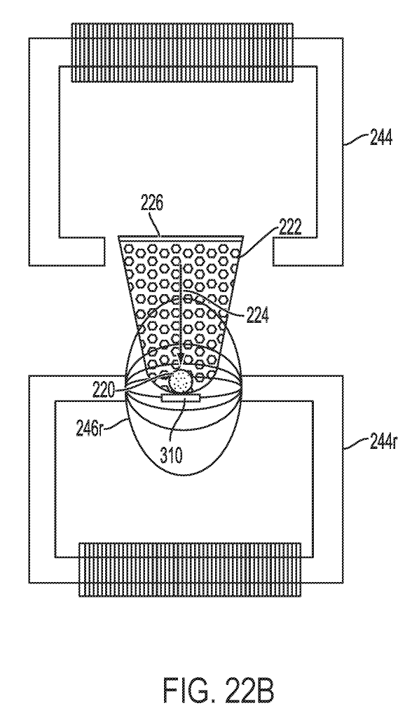

FIGS. 22A and 22B show a configuration analogous to the configuration describe in FIGS. 20A and 20B. In this case a ‘C’ shaped electromagnet is placed both above 244 and below 244r the well 222 and the DC current is switched between the two electromagnets. In FIG. 22A the top electromagnet 244 is energized, its magnetic flux 246 thus drawing the bead 220 up through the reagent solution 224 in the well 222 until it reaches the upper barrier 226. In FIG. 22B the DC current is then switched to the lower magnet 244r and its magnetic flux 246r draws the bead 220 back down through the solution 224 until it hits the bottom of the well 222.

FIGS. 19A, 19B, 20A, 20B, 21A, 21B, 22A and 22B are just examples of possible ways to use the magnetically responsive coated beads. The wells in FIGS. 19A, 19B, 21A and 21B can be dedicated mixing chambers in or out of a cartridge based system or in a dedicated sample processing system. The wells in FIGS. 20A, 20B, 22A and 22B can be horizontally configured wells or vertical or horizontal mixing chambers and in or out of a cartridge based system or in a dedicated sample processing system.

The technology also provides various methods suitable to move the magnetic flux into position to cause the bead to move through the solution in the well or mixing chamber, thus causing mixing. The first method was disclosed in the above discussions of FIGS. 21A, 21B, 22A and 22B which describe how to move the bead through the solution in the well or mixing chamber using an electromagnet with the appropriate core and magnetic flux. The advantages of this method are that it requires no moving parts and a single DC current switched on and off will provide the magnetic flux needed to move the bead. Where space and sufficient power are available, this is an adequate method to move the bead. Other methods of moving the bead will be described below.

For purposes of the following discussion, it will be assumed that moving a magnet also moves the magnetic flux of the magnet, or the magnetic field of the magnet, so that reference to moving a magnet into position to move the beads also refers to moving the magnet's magnetic flux into position to move the beads. This assumption applies to the drawings as well. It will be assumed that magnets in the drawings have a magnetic flux and the magnetic flux will not always be represented in the drawings.

In one aspect of the invention, the magnet is a rare earth magnet, and in particular a neodymium magnet. The size and strength of the magnets used will depend on the available space in which to move the magnet, the size and depth of the well, vessel or mixing chamber, the method used to move the magnet, the orientation of the well, and the orientation of the magnet in relationship to the well.

Generally, the most effective methods of moving the magnet are methods that require very few moving parts with few or no mechanical linkages, that have low voltage and current requirements, and that can be controlled easily with a microcontroller or simple timer circuit. One embodiment disclosed changes the direction of the DC current to move the magnet in and out of position, but simpler embodiments do not require the additional circuitry to accomplish this switching.

All methods disclosed here can be applicable to a vertical, horizontal, or even a diagonal orientation of the reaction well or the mixing chamber. The well or chamber can be either stand alone or in a cartridge based system. The embodiments disclosed herein are not meant to constrain mixing to only one orientation of the reagent well or mixing chamber, or to only stand alone or cartridge based systems, but to include all well/chamber orientations and stand alone or closed systems. A single magnet can be used to actuate one or more beads contained within a single well. In addition, a single magnet can actuate the bead(s) contained within multiple wells/chambers. This can simplify the construction of a system that can run tests within two or more adjacent wells using only a single magnetic source.

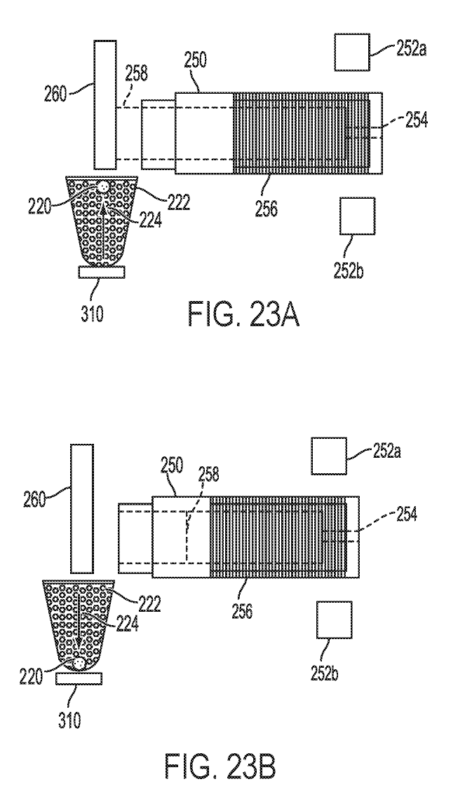

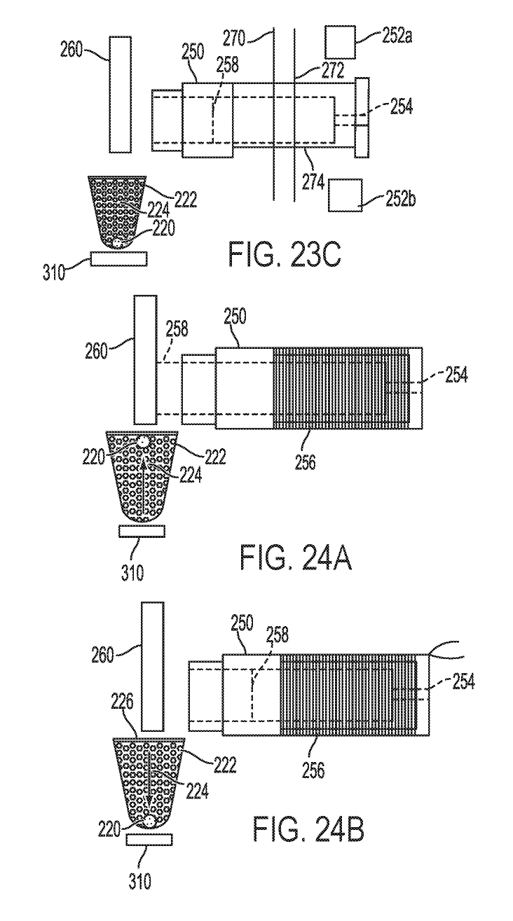

FIGS. 23A, 23B and 23C illustrate one mechanical system for moving the magnets into and out of position. This method uses the magnet 258 to pull the bead 220 up through the solution 224 and allows gravity pull the bead back down through the solution. The magnet is pushed forward by the magnetomotive force generated by the energized coil 256 and drawn back from the well by de-energizing the coil 256 and using the magnetic flux provided by the small magnets 252a and 252b. A non-magnetically responsive material such as aluminum or plastic is used as a barrier 260 to stop the forward motion of the magnet.

FIG. 23A shows the magnet pushed forward by the magnetomotive force generated by the coil 256. Its forward motion has been stopped by the barrier 260 in such a position that it will lift the bead 220 in the well 222 up through the solution 224. FIG. 23B shows the coil 256 de-energized and the magnet 258 pulled back into the bobbin 250 by the attraction of the magnets 252a and 252b, allowing the bead 220 to drop back down through the solution 224 in the well 222. If more rapid mixing were required, the same mechanism described here, or some other method of putting a magnetic flux at the bottom of the well could be used as disclosed in FIGS. 20A, 20B, 22A and 22B.

The system described in FIGS. 23A, 23B and 23C involves designing a plastic bobbin 250 that has two functions. The first is that it be shaped to provide a path for the magnet 258 to travel to and from the position that will allow the bead 220 to be raised and dropped. The second is to hold enough windings of wire so that when the coil 256 is energized with a DC current it will generate enough magnetomotive force to push the magnet forward out of the bobbin. The bobbin also has some relative dimensions and other items that are disclosed in the discussion of FIG. 23C. The method disclosed here uses a single direction DC current that is simply turned on and off with a microcontroller or a simple timing circuit, as would be appreciated by one of ordinary skill in the art. One manner of pulling the magnet 258 back into the bobbin and thus away from the well and the bead is a magnetic flux that is polarized such to attract the magnet 258 and pull it quickly back into the bobbin. The magnetic flux can be provided by one or a plurality of magnets. In the embodiment shown, the magnet flux is provided by two magnets 252a and 252b. The strength, orientation and position of magnets 252a and 252b are important. They must be strong enough to pull the magnet 258 back into the bobbin 250, they must be oriented to attract, rather than repel the magnet 258, and they must be positioned such that their attraction to the magnet 58 can be overcome by the magnetomotive force generated by the energized coil 256.

As stated before, FIG. 23C discloses some relative dimensions and other particulars in the bobbin 250 that allow the back and forth motion to work in this particular embodiment. A vent hole 258 can be positioned at the end of the bobbin 250. This allows air to escape as the magnet 258 is pulled back into the bobbin 250. The center of the coil area 272 must generally be further back on the bobbin then the center of the magnet 270.

As a non-limiting example, the materials and approximate dimensions used to assemble the method disclosed in FIGS. 23A, 23B and 23C are as follows. The plastic bobbin 250 is approximately 1.75 inches long with outside diameters of about 0.6 inches on the large diameters and about 0.5 inches on the small diameters. The internal diameter is about 0.38 inches with a depth of about 1.5 inches. The magnet 258 is a 0.375 inches×1 inch neodymium magnet, and the magnets 252a and 252b are 0.25×0.25 inch neodymium magnets. The coil area 274 (in FIG. 23C) on the bobbin 250 is about 1 inch long. The coil is a winding of 850 turns of #34 magnet wire and is energized by a DC current of 0.5 amps at 12 volts.

The magnets 252a and 252b are encased in a housing that slips over the completed bobbin 250 and holds the magnets 252a and 252b opposite from each other about 0.1875 inches from the side of the coil 256 and about 0.25 inches from the end of the bobbin 250. The barrier 260 is an aluminum block. The “pull up” position of the magnet 258 in FIG. 8a is approximately 0.125 inches past the edge of the well and about 0.125 inches above the well. The switching on and off of the DC current is controlled by a PIC18F1220 microcontroller at up to 5 Hz. This mixing frequency can be easily varied with the firmware, as would be appreciated by one of ordinary skill in the art. The orientation of the magnets 258, 252a and 252b is determined by the direction that the DC current is flowing through the coil 256. Large magnet 258 can be positioned in the bobbin 250 and energize the coil 256. If the magnet 258 is pushed out, then the orientation is correct, if it is pulled in, then either the direction that the DC current is flowing through the coil 256 can be switched, or the magnet 258 can be turned around. Once the large magnet is oriented correctly then it is a simple step to orient the magnets 252a and 252b to hold the large magnet 258 in the bobbin 250.

Another method to move the magnet into position to move the bead in a reaction well or mixing chamber is disclosed in FIGS. 24A and 24B. This method is very similar to the method disclosed in the discussion of FIGS. 23A, 23B and 23C. The primary difference is the removal of the magnets 252a and 252b shown in FIGS. 23A-C, and instead sending the DC current in one direction of the coil 256 to push the magnet 258 out to the “pull up” position as shown in FIG. 24A. Then the direction of the DC current through the coil 256 can be switched to pull the magnet away from the well 222 and bead 220 allowing the bead 220 to drop back through the solution 224 to the bottom of the well 222. Once again, if more rapid mixing were required, the same mechanism described herein, or some other method of putting a magnetic flux at the bottom of the well could be used (for example, the techniques shown in FIGS. 20A, 20B, 22A and 22B).

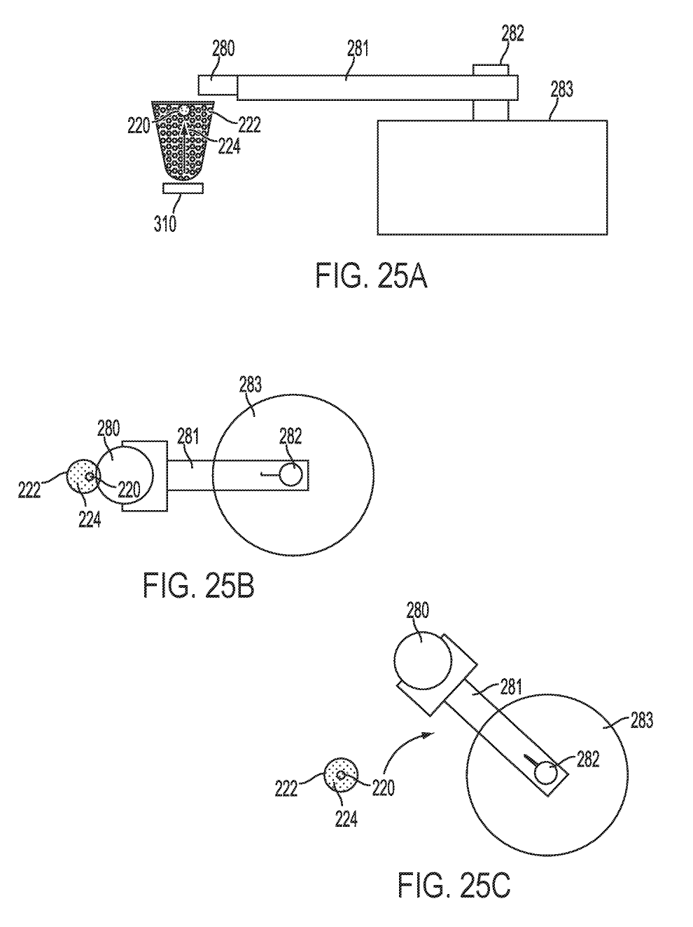

Another method of moving the magnet into position to move the bead in a reaction well or mixing chamber is disclosed in FIGS. 25A-25C. This method employs a rotating solenoid that is controlled with either a single on/off DC current or a Pulse Width Modulated DC current to control the speed of rotation. Again, either a circuit or a microcontroller can be used to control the frequency of the rotation and, in the case of the PWM controlled solenoid, the speed of the rotation. Referring to FIG. 25A, a magnet 280 is attached to an arm 281 that is attached to the armature 282 of a rotating solenoid 283. The magnet used is again a rare earth magnet with sufficient magnetic flux to pull the bead 220 toward it when the magnet is brought into proximity of the well 222 and bead 220. FIG. 25B shows a top view of the rotating solenoid 283 that has been activated by a DC current. When activated, the magnet, attached to the solenoid 283 via the arm 281 and armature 282, is swung over the top of the well 222 in position to move the bead 220 through the solution 224 toward the magnet 280.

FIG. 25C shows the top view of the rotating solenoid 283 that has been de-activated. When de-activated, the magnet, attached to the solenoid 283 via the arm 281 and armature 82, is swung away from the well 222 into a position that allows the bead 220 to drop through the solution 224 toward the bottom of the well 222. Once again, if more rapid mixing were required, the same mechanism described here, or some other method of putting a magnetic flux at the bottom of the well could be used as disclosed in FIGS. 20A, 20B and 22A, 22B.

The methods described here can be used in association with optics systems. As one non-limiting example, FIG. 26 shows the method disclosed in FIG. 23A-23C, 250, 252a, 252b, 254 and 256 attached directly to an optics head 300 that is in position over the reaction well 222 so that readings of florescence levels can be taken during the reaction. The housing used to mount the magnets 252a and 252b is also used to secure the attachment of the bobbin 250 to the optics head 300. The specific housing arrangement is omitted for the sake of clarity. FIG. 27 shows an example of a possible arrangement to accommodate working with an optics head 300 where a rotating solenoid 283 is used to move the magnet 280 in and out of the position to move the bead 220 as disclosed in FIGS. 25A, 25B and 25C. By removing some material 302 from the head 300, the magnet 280 can be swept under the optics systems head 300. Again, the optics head 300 is in position over the reaction well 222 so that readings of florescence levels can be taken during the reaction.

In another example, the optics can be moved away from the reaction well while mixing is occurring and then moved back into position to read florescence levels after mixing is done. In yet another example, the well can be moved away from the optics, the solution can be mixed, and the well can be brought back to the optics position to be read.

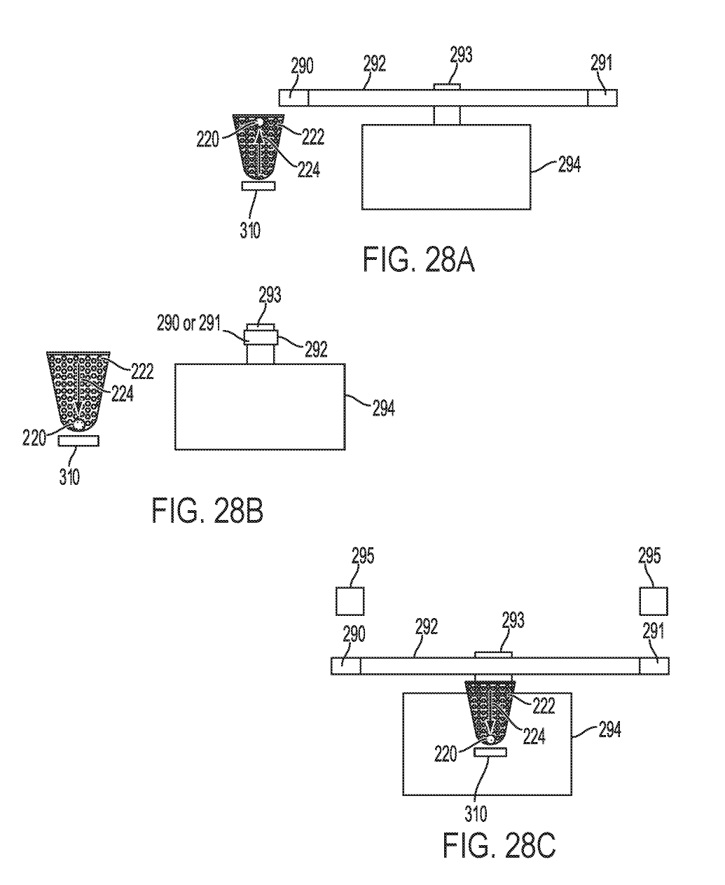

Another method to move the magnet into position to move the bead in a reaction well or mixing chamber is disclosed in FIGS. 28A, 28B and 28C. In this method, an armature 292 is attached to the shaft 293 of an electric motor 294.

Depending on the speed of the motor and the desired mixing frequency, a magnet 290, 291 can be attached at each end of the armature, or as another example, a magnet could be attached at one end 290 and a counterweight 291 attached at the other end of the armature. As the magnet passes over the well (as depicted in FIG. 28A), the bead will be pulled up, and as the magnet is positioned away from the well, the bead will be dropped (FIG. 28B). The position of the armature 292, thus the magnet or magnets 290, 291, when the motor is off can be determined by a position control switch or by placing magnets 295 of sufficient strength and of the opposite polarity of the magnet or magnets 290, 291 on the armature 292 at such a position as to draw the magnets away from the well 222, as shown in FIG. 28C. The armature 292 can be of any shape, including a disk, and can hold a single or a plurality of magnets and counter weights.

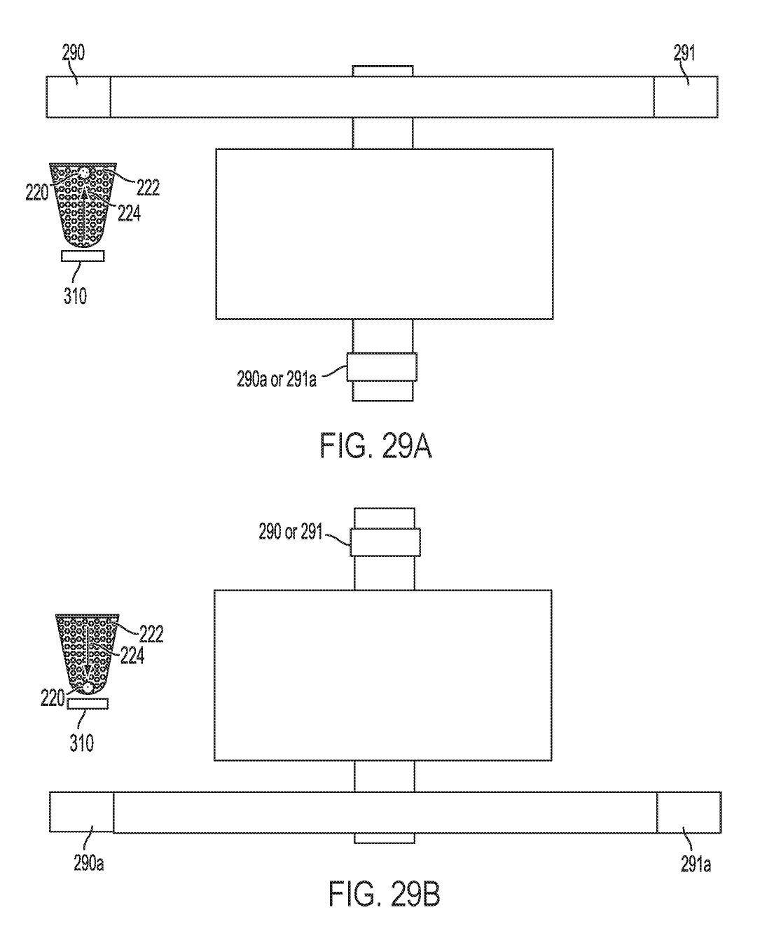

Additionally, FIGS. 29A-29B depict how a secondary armature may be attached to the apparatus of FIGS. 28A-28C wherein the second armature may be positioned below the closed cartridge reaction well and wherein the armature is located at a position being out-of-phase with the first armature. The second armature has additional magnets and counterweights 290a and 291a being embedded therein to provide a secondary magnetic field to the closed cartridge reaction well. The rotation of the shaft then passes the two armatures into their relative positions either above or below the reaction well and draws the bead up and down in a reciprocating fashion in order to achieve the desired mixing.

It is to be understood that the bead can be moved by the magnets in a variety of paths. A simple up-and-down motion can be achieved, or a simple side-to-side motion. In addition, helical patterns can be achieved, circular patterns, etc. The present technology provides a great deal of flexibility of movement of the magnetic bead.

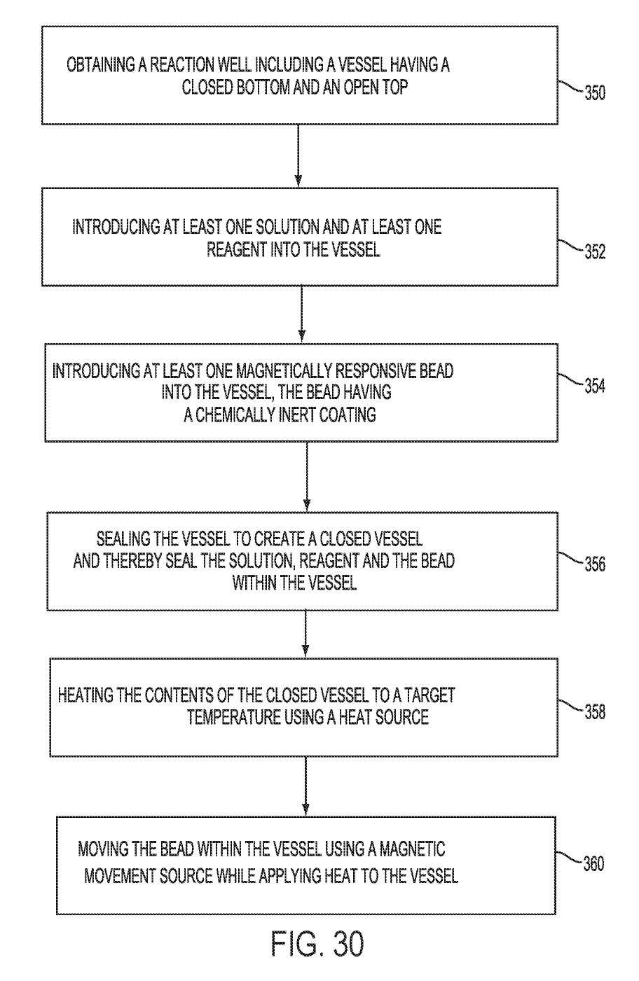

FIG. 30 illustrates one method of providing a homogeneous mixture of solutions and reagents during a heated reaction having a first step 350 including providing a reaction well having a vessel with a closed bottom and an open top. A second step 352 includes providing at least one solution and at least one reagent within the hollow vessel. A third step 354 includes providing at least one magnetically responsive bead having an optical coating and a chemically inert coating into the reaction well. A fourth step 356 includes sealing the reaction well with a barrier that circumvents and seals the open top to form a closed cartridge reaction well containing the solution, reagent and the bead. A fifth step 358 includes heating the contents of the closed cartridge reaction well to a target temperature using a heat source. A sixth step 360 includes moving the bead into an upper portion of the closed cartridge reaction well by oscillating a first magnetic field of a first magnet proximate a first external portion of the closed cartridge reaction well. A seventh step 362 includes moving the bead into a lower portion of the closed cartridge reaction well by oscillating a second magnetic field of a second magnet proximate a second opposing external portion of the closed cartridge reaction well. The method can include the further step of oscillating the first and second magnetic fields out of phase to cause the bead to move in a reciprocating fashion within the closed cartridge reaction well at a sufficient rate that the bead mixes the solution and reagent to have a homogeneous temperature and mixture.

The method can also include discontinuing mixing within the reaction well while the solution is cooled. In this manner, the chemical constituents in the solution that must come into close proximity (or direct contact) with each other, such as an enzyme with its substrate(s), will be allowed to form a reaction. Continual mixing can lower the efficiency of these reactions by preventing the correct location of these reactants, and orientations between them, due to manual agitation. In addition, the mechanical action of the bead will not interfere with reactions within the well that require precise alignment of reactants. Thus, a static liquid system can be established when the chemical reactants require it and a system of liquid movement can be established when rapid thermal transfer is needed by the system.

It should be appreciated that additional steps, as would be recognized by one of ordinary skill in the art, may be employed to utilize each of the specific apparatus embodiments as discussed above.

While the forgoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

1. A sample processing module, comprising:
a PCR assembly, said PCR assembly including at least one PCR reaction vial;
a sample assembly, said sample assembly being configured to hold a fluid sample therein, said sample assembly being attachable to said PCR assembly so as to form a closed system;
a delivery channel formed in said PCR assembly, said delivery channel being in fluidic communication with said PCR reaction vial and with said sample assembly;
a pressurization device, said pressurization device being configured to increase a pressure of said fluid sample in said sample assembly and direct at least a portion said fluid sample through said delivery channel to said PCR reaction vial;
a chemically inert magnetically responsive bead disposed within the PCR reaction vial;
at least a first magnet positioned near a first side of the PCR reaction vial and being configured to provide a first magnetic field through the PCR reaction vial, the first magnetic field being of sufficient strength so as to be capable of moving the magnetically responsive bead within the PCR reaction vial; and
a system for oscillating the strength of the first magnetic field to alter a position of the magnetically responsive bead within the PCR reaction vial.

2. A sample processing module, comprising:
a sample assembly, said sample assembly being configured to hold a fluid sample therein,
said sample assembly including a pressurization device configured to increase a pressure of said fluid sample in said sample assembly; and
a PCR assembly, said PCR assembly including at least one PCR reaction vial, said PCR assembly being configured to receive said sample assembly so as to form a closed system, said PCR assembly including:
a delivery channel, said delivery channel being in fluidic communication with said PCR reaction vial and with said sample assembly;
a vent channel in fluidic communication with said PCR reaction vial;
a flow restriction device;
a sealed vent chamber in fluid communication with said vent channel;
a chemically inert magnetically responsive bead disposed within the PCR reaction vial;
at least a first magnet positioned near a first side of the PCR reaction vial and being configured to provide a first magnetic field through the PCR reaction vial, the first magnetic field being of sufficient strength so as to be capable of moving the magnetically responsive bead within the PCR reaction vial; and
a system for oscillating the strength of the first magnetic field to alter a position of the magnetically responsive bead within the PCR reaction vial.
